(19) United States
(12) Patent Application Publication
(10) Pub. No.: US 2026/0345286 A1
(43) Pub. Date: Nov. 5, 2026

(54) Robotic Surgical Devices, Systems, and Related Methods

(71) Applicant: Virtual Incision Corporation, Lincoln, NE (US)

(72) Inventors: Shane Farritor, Lincoln, NE (US); Chris Santoro, Brooklyn, NY (US); Jeff Shasho, Brooklyn, NY (US); Nishant Kumar, Bergenfield, NJ (US); Mateusz Szczesiak, Forest Hills, NY (US); Jason Herman, East Northport, NY (US)

(21) Appl. No.: 19/738,678

(22) Filed: Jul. 13, 2026

Related U.S. Application Data
(63) Continuation of application No. 18/299,114, filed on Apr. 12, 2023, now Pat. No. 12,690,938.
Application No. 18/299,114 is a continuation of application No. 16/890,424, filed on Jun. 2, 2020, now Pat. No. 11,633,253.
Application No. 16/890,424 is a continuation of application No. 14/212,686, filed on Mar. 14, 2014, now Pat. No. 10,667,883.
(60) Provisional application No. 61/792,508, filed on Mar. 15, 2013.

(51) Int. Cl. A61B 90/35; A61B 1/00; A61B 1/313; A61B 17/29; A61B 17/34; A61B 34/30; A61B 46/10; A61B 90/00; A61B 90/30; A61B 90/50

(52) U.S. Cl. CPC A61B 90/35; A61B 1/00149; A61B 1/3132; A61B 17/29; A61B 17/3423; A61B 34/30; A61B 46/10; A61B 90/50; A61B 2034/302; A61B 2034/305; A61B 90/30; A61B 90/361

(57) ABSTRACT

The embodiments disclosed herein relate to various medical device components, including components that can be incorporated into robotic and/or in vivo medical devices. Certain embodiments include various modular medical devices for in vivo medical procedures.

### CROSS-REFERENCE TO RELATED APPLICATIONS

This application claims priority as a continuation to U.S. application Ser. No. 18/299,114, filed Apr. 12, 2023, and entitled “Robotic Surgical Devices, Systems, and Related Methods,” which claims priority as a continuation to U.S. application Ser. No. 16/890,424, filed Jun. 2, 2020, and entitled “Robotic Surgical Devices, Systems, and Related Methods,” which issued as U.S. Pat. No. 11,633,253 on Apr. 25, 2023, which claims priority as a continuation to U.S. application Ser. No. 14/212,686, filed Mar. 14, 2014, and entitled “Robotic Surgical Devices, Systems, and Related Methods,” which issued as U.S. Pat. No. 10,667,883 on Jun. 2, 2020, which claims the benefit under 35 U.S.C. § 119(e) to U.S. Provisional Application 61/792,508, filed Mar. 15, 2013, and entitled “Robotic Surgical Devices, Systems and Methods,” all of which are hereby incorporated herein by reference in their entireties.

### TECHNICAL FIELD

The embodiments disclosed herein relate to various medical devices and related components, including robotic and/or in vivo medical devices and related components. Certain embodiments include various robotic medical devices, including robotic devices that are disposed within a body cavity and positioned using a support component disposed through an orifice or opening in the body cavity. Further embodiment relate to methods of operating the above devices.

### BACKGROUND

Invasive surgical procedures are essential for addressing various medical conditions. When possible, minimally invasive procedures such as laparoscopy are preferred.

However, known minimally invasive technologies such as laparoscopy are limited in scope and complexity due in part to 1) mobility restrictions resulting from using rigid tools inserted through access ports, and 2) limited visual feedback. Known robotic systems such as the da Vinci® Surgical System (available from Intuitive Surgical, Inc., located in Sunnyvale, CA) are also restricted by the access ports, as well as having the additional disadvantages of being very large, very expensive, unavailable in most hospitals, and having limited sensory and mobility capabilities.

There is a need in the art for improved surgical methods, systems, and devices.

### BRIEF DESCRIPTION OF THE DRAWINGS

FIG. 1A is a side view of a robotic surgical device according to one embodiment.

FIG. 1B is perspective front view of the device of FIG. 1.

FIG. 1C is a perspective view of the device of FIG. 1.

FIG. 1D is an end view of the device of FIG. 1.

FIG. 2A is a cutaway view of the interior body and shoulder of the robotic medical device, according to one embodiment.

FIG. 2B is a rotated cutaway view of the robotic medical device of FIG. 2A.

FIG. 2C is a perspective cutaway view of the medical device, according to the embodiment of FIG. 2A.

FIG. 2D is a further cutaway perspective view of the medical device body, according to the embodiment of FIG. 2A.

FIG. 2E is a cutaway view of the lower body and shoulder of a robotic device, according to the embodiment of FIG. 2A.

FIG. 3A is a cutaway side view of the upper arm of the robotic medical device, according to one embodiment.

FIG. 3B is a perspective view of the embodiment of FIG. 3A.

FIG. 3C is a different perspective view of the embodiment of FIG. 3A.

FIG. 3D is a reverse perspective view of the embodiment of FIG. 3A.

FIG. 3E is an alternate perspective view of medical device as depicted in FIG. 3D.

FIG. 4A is a cutaway view of the internal components of the right upper arm of a robotic device, according to one embodiment.

FIG. 4B is a rotated sideview of the embodiment of FIG. 4A.

FIG. 4C is a further rotated sideview of the embodiment of FIG. 4A.

FIG. 4D is an endlong view of the embodiment of FIG. 4A.

FIG. 4E is a further endlong view of the embodiment of FIG. 4A.

FIG. 5A is a endlong view of the lower arm of a robotic device, according to one embodiment.

FIG. 5B is cutaway sideview of the internal components of the lower arm of the embodiment of FIG. 5A along line A-A.

FIG. 5C is cutaway sideview of the internal components of the lower arm of the embodiment of FIG. 5A along line B-B.

FIG. 6A is a cross-sectional view of the end effector portion of the forearm depicting the electrical portions, according to an exemplary embodiment.

FIG. 6B is a top perspective view of external view of complimentary portion of the forearm to the embodiment of FIG. 6A.

FIG. 7 is a bottom perspective schematic of the internal components of the lower arm of a robotic device, according to one embodiment.

FIG. 8A is cutaway sideview of the internal components of the lower arm of the embodiment of FIG. 5A along line A-A, detailing further electronic components.

FIG. 8B is a close view of the section C-C of the embodiment of FIG. 8A.

FIG. 9A is a perspective view of the exterior of the forearm according to one embodiment.

FIG. 9B is an internal view perspective of the embodiment of FIG. 9A

FIG. 10A is a perspective view of one embodiment of the robotic device comprising an inner fluidic seal.

FIG. 10B is a perspective view of the embodiment of FIG. 10A further comprising further outer fluidic seal.

FIG. 11A is a side cutaway view of one embodiment of a rigid-flex PCB component within the forearm of the device.

FIG. 11B is a further perspective view of the embodiment of FIG. 11A.

FIG. 12A depicts a top view of a robotic device during insertion, according to one embodiment.

FIG. 12B is a front view of the device of FIG. 12A.

FIG. 12C is a side view of the device of FIG. 12A.

FIG. 12D is a perspective view of the device of FIG. 12A.

FIG. 13A depicts a top view of a robotic device during insertion, according to one embodiment.

FIG. 13B is a front view of the device of FIG. 13A.

FIG. 13C is a side view of the device of FIG. 13A.

FIG. 13D is a perspective view of the device of FIG. 13A.

FIG. 14A depicts a top view of a robotic device during insertion, according to one embodiment.

FIG. 14B is a front view of the device of FIG. 14A.

FIG. 14C is a side view of the device of FIG. 14A.

FIG. 14D is a perspective view of the device of FIG. 14A.

FIG. 15A depicts a top view of a robotic device during insertion, according to one embodiment.

FIG. 15B is a front view of the device of FIG. 15A.

FIG. 15C is a side view of the device of FIG. 15A.

FIG. 15D is a perspective view of the device of FIG. 15A.

FIG. 16A depicts a top view of a robotic device during insertion, according to one embodiment.

FIG. 16B is a front view of the device of FIG. 16A.

FIG. 16C is a side view of the device of FIG. 16A.

FIG. 16D is a perspective view of the device of FIG. 16A.

FIG. 17A depicts a top view of a robotic device during insertion, according to one embodiment.

FIG. 17B is a front view of the device of FIG. 17A.

FIG. 17C is a side view of the device of FIG. 17A.

FIG. 17D is a perspective view of the device of FIG. 17A.

### DETAILED DESCRIPTION

The various systems and devices disclosed herein relate to devices for use in medical procedures and systems. More specifically, various embodiments relate to various medical devices, including robotic devices and related methods and systems. Certain implementations relate to such devices for use in laparo-endoscopic single-site (LESS) surgical procedures.

It is understood that the various embodiments of robotic devices and related methods and systems disclosed herein can be incorporated into or used with any other known medical devices, systems, and methods. It is understood that the various embodiments of robotic devices and related methods and systems disclosed herein can be incorporated into or used with any other known medical devices, systems, and methods. For example, the various embodiments disclosed herein may be incorporated into or used with any of the medical devices and systems disclosed in copending U.S. applications Ser. No. 11/766,683 (filed on Jun. 21, 2007 and entitled “Magnetically Coupleable Robotic Devices and Related Methods”), Ser. No. 11/766,720 (filed on Jun. 21, 2007 and entitled “Magnetically Coupleable Surgical Robotic Devices and Related Methods”), Ser. No. 11/966,741 (filed on Dec. 28, 2007 and entitled “Methods, Systems, and Devices for Surgical Visualization and Device Manipulation”), 61/030,588 (filed on Feb. 22, 2008), Ser. No. 12/192,663 (filed Aug. 15, 2008 and entitled Medical Inflation, Attachment, and Delivery Devices and Related Methods”), Ser. No. 12/192,779 (filed on Aug. 15, 2008 and entitled “Modular and Cooperative Medical Devices and Related Systems and Methods”), 61/640,879 (filed on May 1, 2012), Ser. No. 13/493,725 (filed Jun. 11, 2012 and entitled “Methods, Systems, and Devices Relating to Surgical End Effectors”), Ser. No. 13/546,831 (filed Jul. 11, 2012 and entitled “Robotic Surgical Devices, Systems, and Related Methods”), 61/680,809 (filed Aug. 8, 2012), Ser. No. 13/573,849 (filed Oct. 9, 2012 and entitled “Robotic Surgical Devices, Systems, and Related Methods”), and Ser. No. 13/738,706 (filed Jan. 10, 2013 and entitled “Methods, Systems, and Devices for Surgical Access and Insertion”), and U.S. Patents 7,492,116 (filed on Apr. 3, 2007 and entitled “Robot for Surgical Applications”), 7,772,796 (filed on Nov. 29, 2007and entitled “Robot for Surgical Applications”), 8,179,073 (issued May 15, 2012, and entitled “Robotic Devices with Agent Delivery Components and Related Methods”), 8,343,171 (filed on Jul. 11, 2008 and entitled “Methods and Systems of Actuation in Robotic Devices”), and 8,679,096 (filed Nov. 26, 2008 and entitled “Multifunctional Operational Component for Robotic Devices”), all of which are hereby incorporated herein by reference in their entireties.

Certain device and system implementations disclosed in the applications listed above can be positioned within a body cavity of a patient in combination with a support component similar to those disclosed herein. An “in vivo device” as used herein means any device that can be positioned, operated, or controlled at least in part by a user while being positioned within a body cavity of a patient, including any device that is coupled to a support component such as a rod or other such component that is disposed through an opening or orifice of the body cavity, also including any device positioned substantially against or adjacent to a wall of a body cavity of a patient, further including any such device that is internally actuated (having no external source of motive force), and additionally including any device that may be used laparoscopically or endoscopically during a surgical procedure. As used herein, the terms “robot,” and “robotic device” shall refer to any device that can perform a task either automatically or in response to a command from an external console or control system, as has been described previously.

Certain embodiments provide for insertion of the present invention into the cavity while maintaining sufficient insufflation of the cavity. Further embodiments minimize the physical contact of the surgeon or surgical users with the present invention during the insertion process. Other implementations enhance the safety of the insertion process for the patient and the present invention. For example, some embodiments provide visualization of the present invention as it is being inserted into the patient's cavity to ensure that no damaging contact occurs between the system/device and the patient. In addition, certain embodiments allow for minimization of the incision size/length. Further implementations reduce the complexity of the access/insertion procedure and/or the steps required for the procedure. Other embodiments relate to devices that have minimal profiles, minimal size, or are generally minimal in function and appearance to enhance ease of handling and use.

Certain implementations disclosed herein relate to “combination” or “modular” medical devices that can be assembled in a variety of configurations. For purposes of this application, both “combination device” and “modular device” shall mean any medical device having modular or interchangeable components that can be arranged in a variety of different configurations. The modular components and combination devices disclosed herein also include segmented triangular or quadrangular-shaped combination devices. These devices, which are made up of modular components (also referred to herein as “segments”) that are connected to create the triangular or quadrangular configuration, can provide leverage and/or stability during use while also providing for substantial payload space within the device that can be used for larger components or more operational components. As with the various combination devices disclosed and discussed above, according to one embodiment these triangular or quadrangular devices can be positioned inside the body cavity of a patient in the same fashion as those devices discussed and disclosed above.

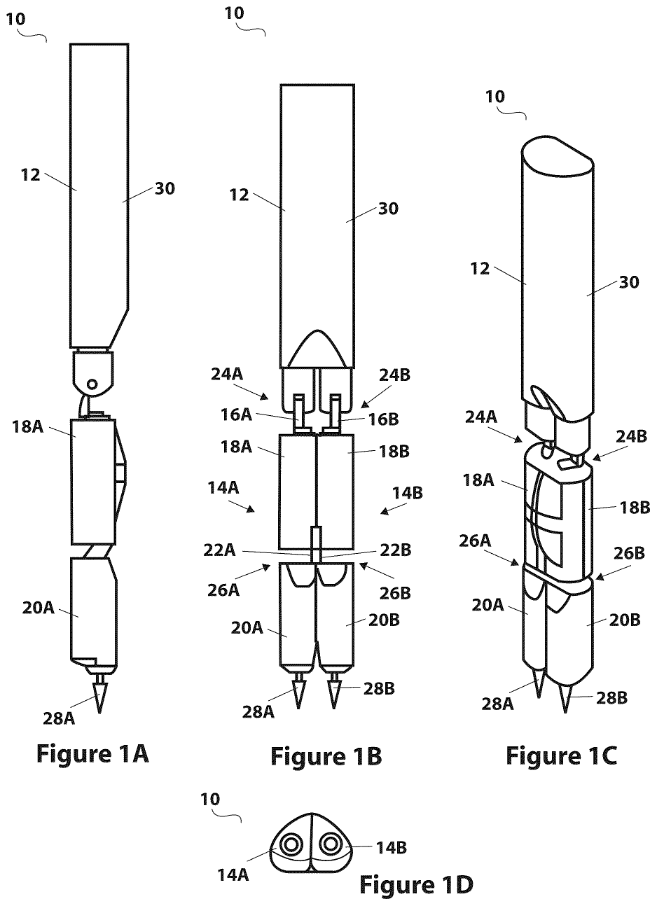

As shown generally in FIGS. 1A, 1B, 1C, and 1D, certain exemplary embodiments relate to a device 10 having a body 12 with two arms 14A, 14B operably coupled thereto. The body 12 as shown further comprises a casing 30. The body 12 is also referred to as a “device body.” Each arm 14A, 14B has a first coupling link 16A, 16B that couples the arm 14A, 14B to the body 12.

As is best shown in FIGS. 1B-1C, this first coupling link 16A, 16B can also be referred to herein as a “first coupling component” or “shoulder link” and is part of the first rotatable joint 24A, 24B (also referred to herein as the “shoulder joint”). Each arm 14A, 14B has an upper arm (also referred to herein as an “inner arm,” “inner arm assembly,” “inner link,” “inner link assembly,” “upper arm assembly,” “first link,” or “first link assembly”) 18A, 18B, and a forearm (also referred to herein as an “outer arm,” “outer arm assembly,” “outer link,” “outer link assembly,” “forearm assembly,” “second link,” or “second link assembly”) 20A, 20B.

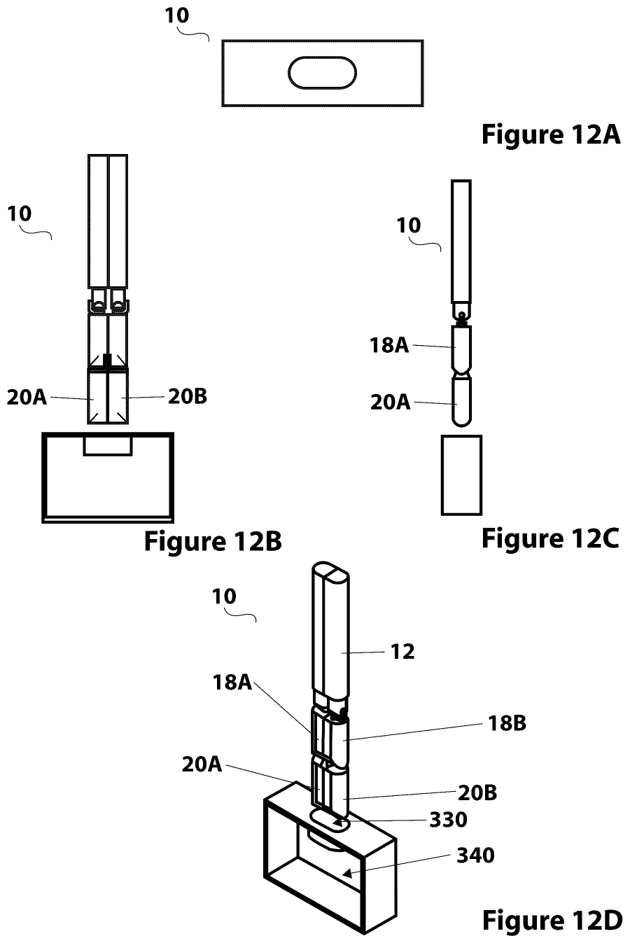

As is shown in FIGS. 1A-1C and further discussed in relation to FIGS. 12-17 below, the upper arms 18A, 18B are rotatably coupled to the coupling links 16A, 16B, which are rotatably coupled to the body 12. Each arm 14A, 14B has a second coupling link 22A, 22B that couples the upper arm 18A, 18B to the forearm 20A, 20B. This second coupling link 22A, 22B can also be referred to herein as a “second coupling component” or “elbow link” and is part of the second rotatable joint 26A, 26B (also referred to herein as the “elbow joint”). More specifically, in the right arm 14A, the upper arm 18A is rotatably coupled to the forearm 20A at the elbow joint 26A via the elbow link 22A, while in the left arm 14B, the upper arm 18B is rotatably coupled to the forearm 20B at the elbow joint 26B via elbow link 22B.

As shown, each of the arms 14A, 14B also has an end effector 28A, 28B operably coupled to the distal end of the forearm 20A, 20B. An end effector can also be referred to herein as an “operational component.”

In one implementation, each of the arms 14A, 14B has six degrees of freedom. That is, as explained in further detail below, each arm 14A, 14B has three degrees of freedom at the shoulder, one degree of freedom at the elbow, and two degrees of freedom at the end effector (which can be rotated end effector roll- and opened/closed). As such, the six degrees of freedom of each arm 14A, 14B are analogous to the degrees of freedom of a human arm, which also has three degrees of freedom at the shoulder and one at the elbow. One advantage of an arm having four degrees of freedom (with an end effector having two degrees of freedom) is that the end effector can have multiple orientations at the same Cartesian point. This added dexterity allows the surgeon or other user more freedom and a more intuitive sense of control while operating the device.

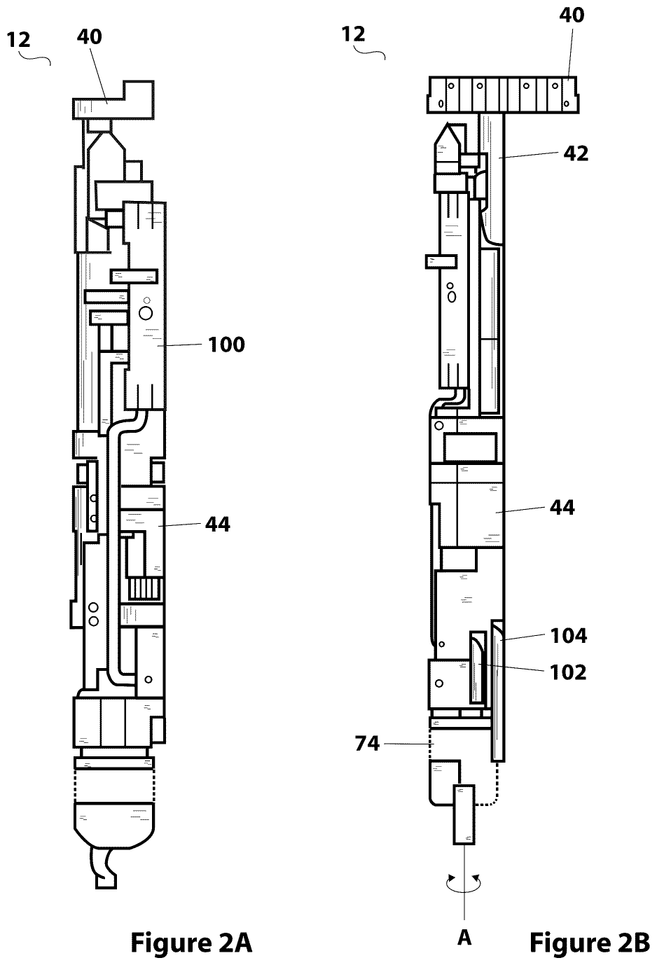

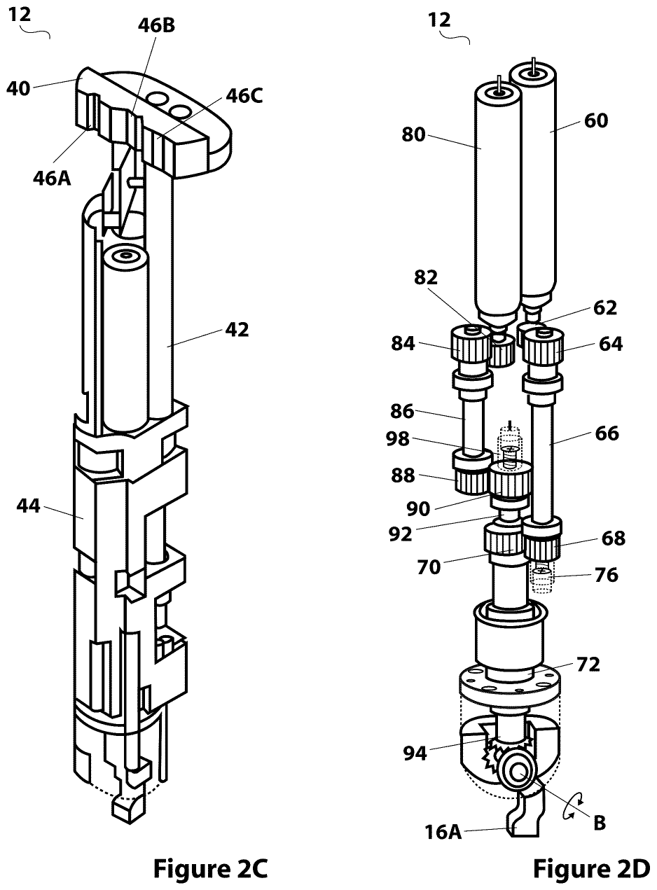

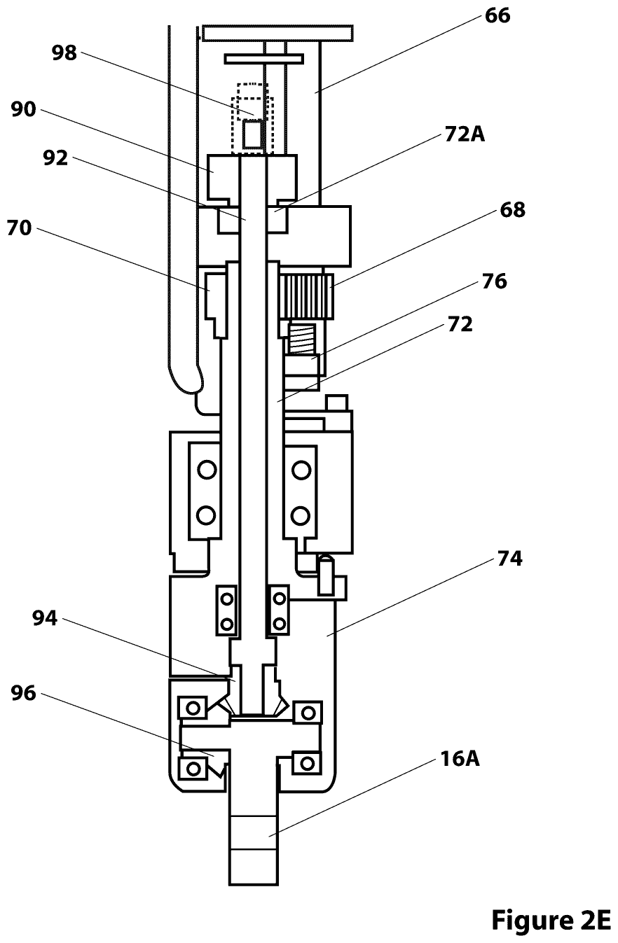

The internal components of the body 12 are depicted in the various embodiments shown in FIGS. 2A, 2B, 2C, 2D, and 2E. The body 12 is shown in these figures without its casing 30. More specifically, these figures depict the right half of the body 12 and the internal components that control/actuate the right arm 14A. It is to be understood that the internal components in the left half (not shown) that operate/control/actuate the left arm 14B are substantially the same as those depicted and described herein and that the descriptions provided below apply equally to those components as well.

FIGS. 2A, 2B, and 2C include the internal structural or support components of the body 12. In one implementation, the body 12 has an internal top cap 40, an internal support rod 42, and an internal support chassis 44, as shown. The support rod 42 couples the top cap 40 to the support chassis 44. In certain embodiments, the support chassis comprises an aluminum structure. In alternate embodiments, an injection-molded polymer may be used. These components maintain the structure of the body 12 and provide structural support for the components disposed therein, and in certain embodiments are surrounded by a housing or shell. According to one embodiment, the internal top cap 40 defines three partial lumens 46A, 46B, 46C as best shown in FIG. 2C. The top cap 40 couples to the body casing 30 such that each of the partial lumens 46A, 46B, 46C is formed into a full lumen defined by the coupling of the cap 40 and casing 30. As will be described in further detail below, these lumens 46A, 46B, 46C can be configured to receive various wires, cords, or other components to be inserted into or through the body 12.

In contrast to FIGS. 2A-2C, FIG. 2D depicts the internal actuation and control components of the right half of the body 12 with the internal structural or support components hidden in order to better display the internal actuation and control components. These internal actuation and control components are configured to provide two degrees of freedom at the shoulder joint 24A.

FIG. 2E is an enlarged view of the distal end of the body 12. In one embodiment, certain of the internal components depicted in FIGS. 2D and 2E are configured to actuate rotation at the shoulder joint 24A around axis A (as best shown in FIG. 2B), which is parallel to the longitudinal axis of the body 12. This rotation around axis A is also referred to as “yaw” or “shoulder yaw.” The rotation, in one aspect, is created as follows. An actuator 60 is provided that is, in this implementation, a motor assembly 60. The motor assembly 60 is operably coupled to the proximal motor gear 62, which is coupled to the proximal driven gear 64 such that rotation of the proximal motor gear 62 causes rotation of the proximal driven gear 64. The proximal driven gear 64 is fixedly coupled to a proximal transmission shaft 66, which has a distal transmission gear 68 at the opposite end of the shaft 66. The distal transmission gear 68 is coupled to a distal driven gear 70, which is fixedly coupled to the distal transmission shaft 72. A magnet holder 76 containing a magnet is also operably coupled to the distal transmission gear 68. The holder 76 and magnet are operably coupled to a magnetic encoder (not shown).

It is understood that the magnet holder 76, magnet, and magnetic encoder (and those similar components as discussed elsewhere herein in relation to other joints) are components of an absolute position sensor that is the same as or substantially similar to one or more of the absolute position sensors disclosed in U.S. application Ser. Nos. 13/573,849 filed Oct. 9, 2012, and 13/833,605 filed Mar. 15, 2013, which are hereby incorporated by reference in their entirety. The distal transmission shaft 72 is fixedly coupled at its distal end to a rotatable pitch housing 74 (as best shown in FIGS. 2B and 2E) such that rotation of the distal driven gear 70 causes rotation of the shaft 72 and thus rotation of the housing 74 around axis A as shown in FIG. 2B.

According to one implementation, certain other internal components depicted in FIG. 2D are configured to actuate rotation at the shoulder joint 24A around axis B (as best shown in FIG. 2D), which is perpendicular to the longitudinal axis of the body 12. This rotation around axis B is also referred to as “pitch” or “shoulder pitch.” The rotation, in one embodiment, is created as follows. An actuator 80 is provided that is, in this implementation, a proximal shoulder motor assembly 80. The motor assembly 80 is operably coupled to a proximal shoulder motor gear 82, which is coupled to the proximal shoulder driven gear 84 such that rotation of the proximal shoulder motor gear 82 causes rotation of the proximal shoulder driven gear 84. This driven gear 84 is fixedly coupled to a proximal shoulder transmission shaft 86, which has a proximal shoulder transmission gear 88 at the opposite end of the shaft 86.

The proximal transmission gear 88 is coupled to a distal shoulder driven gear 90, which is fixedly coupled to the distal shoulder shaft 92. A magnet holder 98 containing a magnet is also operably coupled to the driven gear 90. The holder 98 and magnet are operably coupled to a magnetic encoder (not shown). As best shown in FIG. 2E, a portion of the distal shoulder shaft 92 is disposed within the lumen 72A of the shaft 72 described above and extends out of the distal end of the shaft 72 into the housing 74. As best shown in FIG. 2E, the distal end of the shaft 92 is coupled to a rotation gear 94 that is a bevel gear 94. The rotation gear 94 is operably coupled to link gear 96, which is also a bevel gear 96 according to one implementation. The link gear 96 is operably coupled to the shoulder link 16A (discussed above) such that rotation of the shaft 92 causes rotation of the rotation gear 94 and thereby the rotation of the link gear 96 and thus rotation of the link 16A around axis B as best shown in FIG. 2D.

In this embodiment, the two axes of rotation are coupled. That is, if solely rotation around axis A (pure yaw) is desired, then the “pitch drive train” (the motor 80 and all coupled gears and components required to achieve rotation around axis B) must match the speed of the “yaw drive train” (the motor 60 and all coupled gears and components required to achieve rotation around axis A) such that there is no relative angular displacement between the pitch housing 74 and the rotation gear 94. In contrast, if solely rotation around axis B (pure pitch) is desired, then the yaw drive train must hold position while the pitch drive train is actuated.

In one implementation as shown in FIG. 2A, the body 12 has a rigid-flex PCB 100 positioned in the body. The PCB 100 is operably coupled to and communicates with the motors 60, 80 and magnetic encoders (not shown) to perform the yaw and pitch functions.

According to another embodiment, at least one connection component is associated with the body 12. More specifically, in this implementation, a power/communication line 102 and a cautery power line 104 are coupled at their proximal ends to one or more external power sources (not shown) and extend into the device 10 through one or more of the three lumens 46A, 46B, 46C defined partially by internal top cap 40. The lines 102, 104 extend through the body 12 and exit as shown in FIG. 2B and extend to the upper arm segment. In certain embodiments, the lines 102, 104 are not continuous, but occur in series. In certain of these embodiments, the lines contain terminus at various PCB boards. In yet further embodiments of the lines may run in parallel.

In one embodiment, the body 12 can be coupled at its proximal end to a positioning rod (also referred to as an “insertion rod”) (not shown). It is understood that the positioning rod can be any such known component for helping to position the device 10 and/or maintain and stabilize the position of the device 10. According to one implementation, the power/communication line 102 and/or the cautery power line 104 can extend proximally through one or more lumens in the positioning rod.

In one embodiment, any of the motors discussed and depicted herein can be brush or brushless motors, such as brushless DC motors. Further, the motors can be, for example, 6 mm, 8 mm, or 10 mm diameter motors. Alternatively, any known size that can be integrated into a medical device can be used. In a further alternative, the actuators can be any known actuators used in medical devices to actuate movement or action of a component. Examples of motors that could be used for the motors described herein include the EC 10 BLDC+GP10A Planetary Gearhead, EC 8 BLDC+GP8A Planetary Gearhead, or EC 6 BLDC+GP6A Planetary Gearhead, all of which are commercially available from Maxon Motors, located in Fall River, MA.

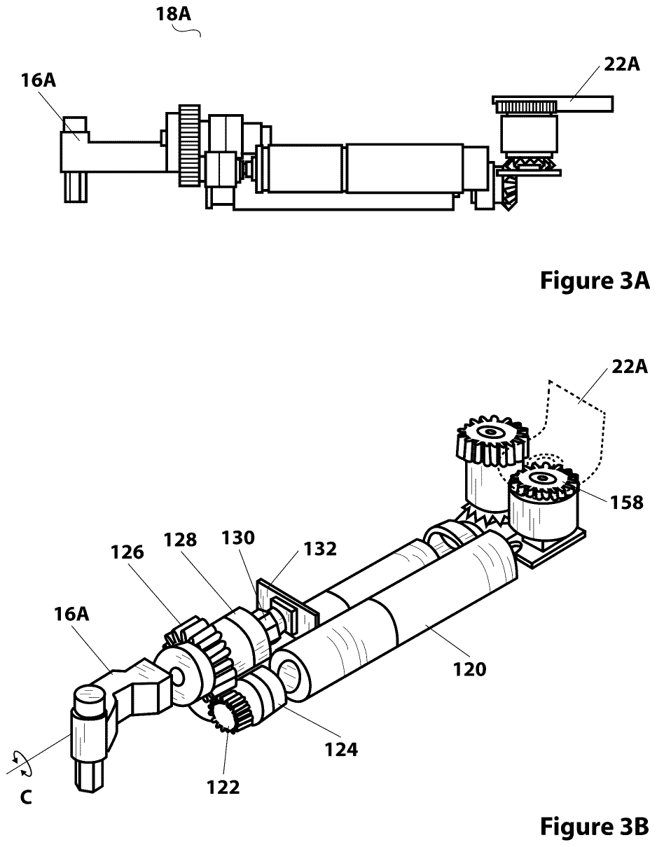

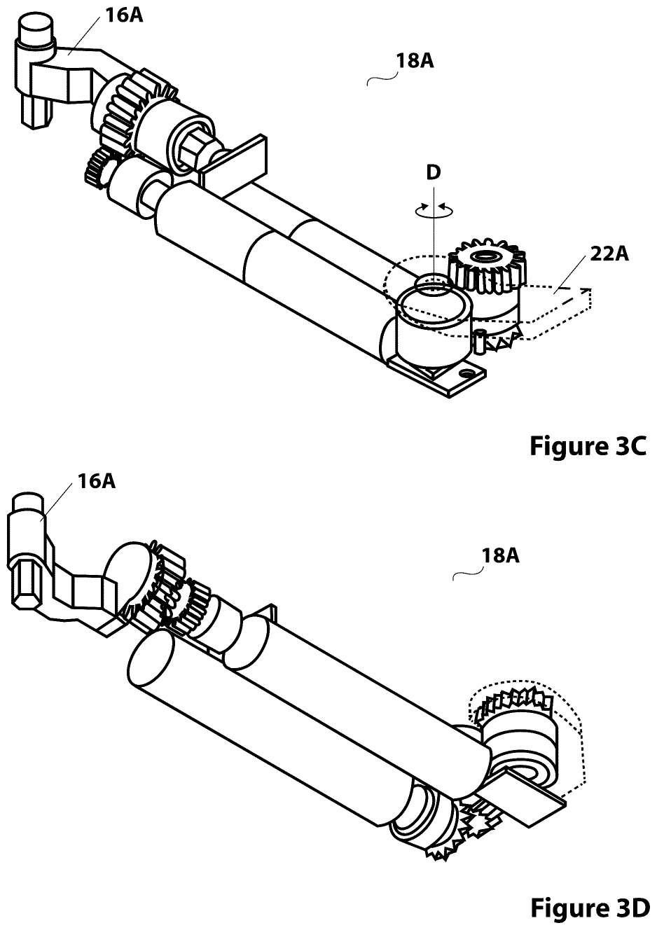

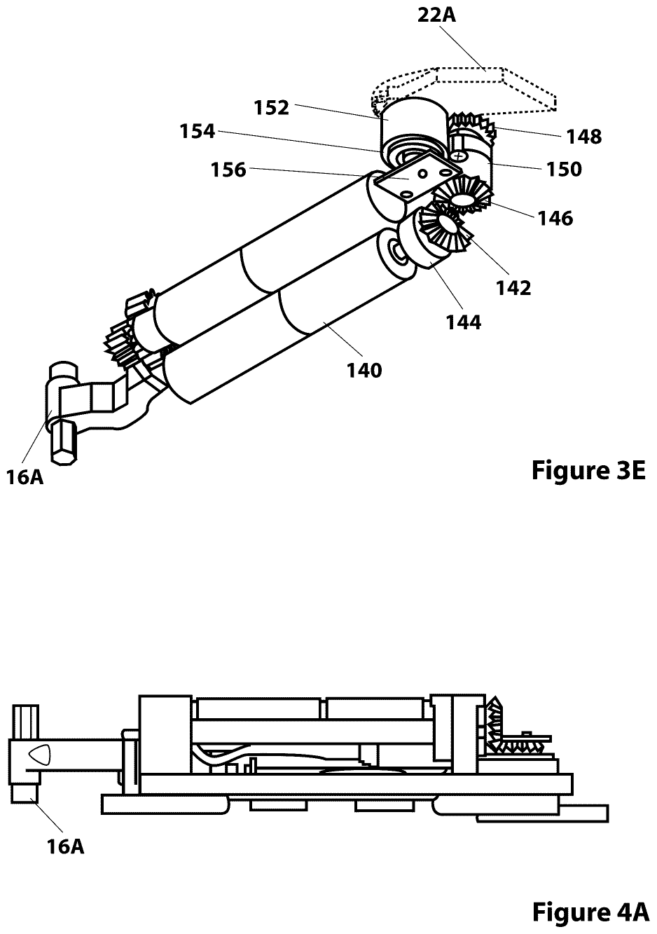

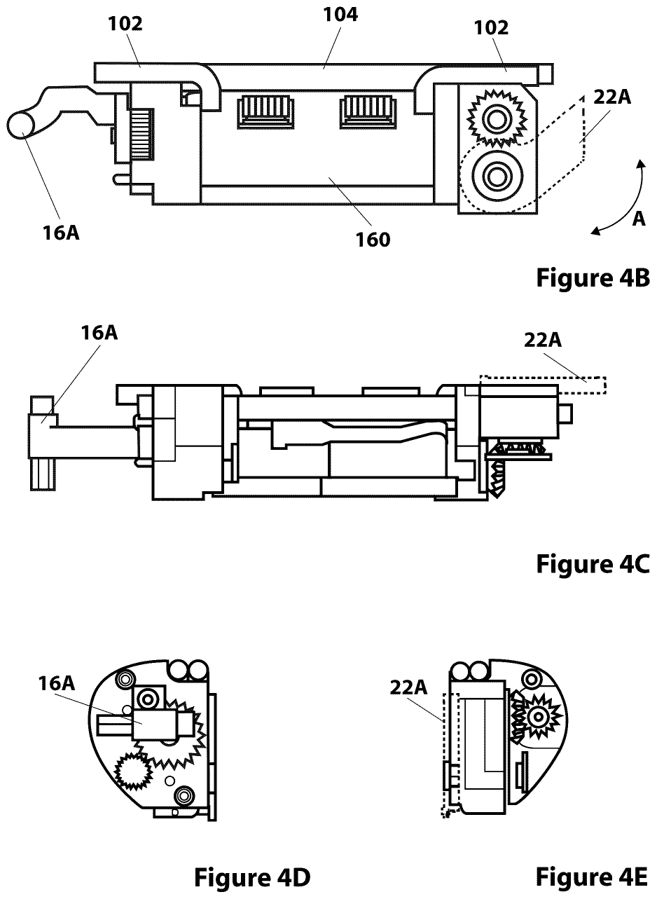

FIGS. 3A, 3B, 3C, 3D, 3E, 4A, 4B, 4C, 4D, and 4E according to one embodiment, depict the internal components of the right upper arm 18A, which is shown in these figures without its casing. More specifically, these figures depict the right arm 14A and the internal components therein. It is understood that the internal components in the left upper arm 18B are substantially the same as those depicted and described herein and that the descriptions provided below apply equally to those components as well.

FIGS. 3A-3E depict the internal components of the right upper arm 18A, including actuators, drive components, and electronics, with the internal structural or support components hidden in order to better display the internal components. In contrast to FIGS. 3A-3E, FIGS. 4A-4E include both the internal actuator, drive, and electronics components, but also the internal structural or support components of the right upper arm 18A.

In one embodiment, certain of the internal components depicted in FIGS. 3A-3E are configured to actuate rotation at the shoulder link 16A around axis C (as best shown in FIG. 3B), which is parallel to the longitudinal axis of the right upper arm 18A. This rotation around axis C is also referred to as “shoulder roll.” The rotation, in one aspect, is created as follows: a first shoulder actuator 120 is provided that is, in this implementation, a motor assembly 120. This motor assembly 120 is operably coupled to a first shoulder motor gear 122. This motor gear 122 is supported by a first shoulder bearing pair 124. This motor gear 122 is coupled to the shoulder driven gear 126 such that rotation of the first shoulder motor gear 122 causes rotation of the driven gear 126. The driven gear 126 is fixedly coupled to the shoulder link 16A such that rotation of the driven gear 126 causes rotation of the shoulder link 16A around axis C as shown in FIG. 3B. The driven gear 126 is supported by a second bearing pair 128. A magnet holder 130 further comprising a magnet is also operably coupled to the driven gear 126. The holder 130 and magnet are operably coupled to a magnetic encoder 132.

The rotation of the shoulder link 16A around axis C causes the right upper arm 18A (and thus the forearm 20A) to rotate in relation to the body 12. According to one embodiment, this rotation adds an additional degree of freedom not provided in prior two-armed surgical devices.

According to one implementation, certain of the internal components depicted in FIGS. 3A-3E are configured to actuate rotation at the elbow link 22A around axis D (as best shown in FIG. 3C), which is perpendicular to the longitudinal axis of the right upper arm 18A. This rotation around axis D is also referred to as “elbow yaw.” The rotation, in one aspect, is created as follows. An actuator 140 is provided that is, in this implementation, a second upper arm motor assembly 140. This motor assembly 140 is operably coupled to the second upper arm motor gear 142, which is a beveled gear in this embodiment. This motor gear 142 is supported by a bearing 144. The motor gear 142 is coupled to the driven gear 146 such that rotation of the motor gear 142 causes rotation of the driven gear 146. The driven gear 146 is fixedly coupled to a link gear 148, which is coupled to the gear teeth 158 (as best shown in FIG. 3B) of the elbow link 22A such that rotation of the driven gear 146 causes rotation of the elbow link 22A around axis D as shown in FIG. 3C. The driven gear 146 and link gear 148 are supported by a bearing pair 150. Further, the elbow link 22A is supported by a bearing pair 152. A magnet holder 154 containing a magnet is also operably coupled to the elbow link 22A. The holder 154 and magnet are operably coupled to a magnetic encoder 156.

According to one embodiment, the additional coupling of the link (or mesh) gear 148 and the elbow link 22A can provide certain advantages, including an additional external reduction (because the gear 148 has fewer gear teeth than the elbow link 22A), shortening of the upper arm 18A and improved joint range of motion. In various embodiments, as with the embodiment shown in FIGS. 4A-E, the robotic devices represent an improvement in range of motion of the elbow joint by reducing the relative distance between the center of the rotational center of the elbow link 22A and the desired direction of travel and preventing physical impediment (as is depicted by arrow A in FIG. 2B).

As shown in FIG. 4B, the upper arm 18A can have a rigid-flex PCB 160 positioned therein. In one embodiment, the PCB 160 is operably coupled to and communicate with the actuators 120, 140 and magnetic encoders 132, 156.

According to another embodiment, at least one connection component is associated with the upper arm 18A. More specifically, in this implementation, the power/communication line 102 and the cautery power line 104 enter through a port (not shown) at the proximal end of the upper arm 18A and exit through a port (not shown) at the distal end.

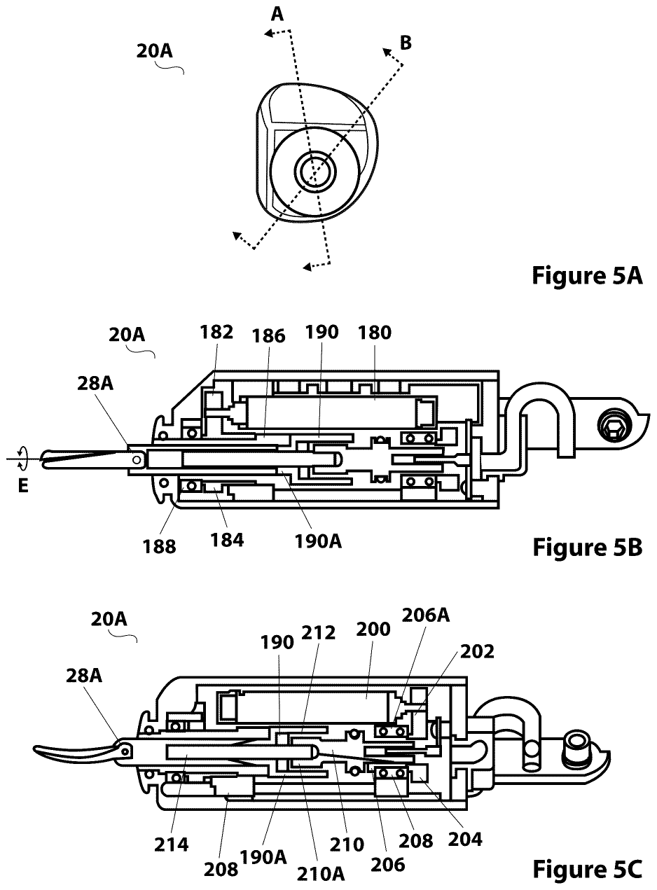

FIGS. 5A-9B depict various embodiments of a right forearm 20A. The various implementations disclosed and depicted herein include the actuators, drive components, and electronics that can be used to accomplish both tool roll and tool drive (open/close action), as will be described in further detail below. As set forth below, the forearm 20A also has two electrically isolated cautery circuits, enabling both bipolar and monopolar cautery end effectors. Certain embodiments are configured to allow for easy removal and replacement of an end effector (a “quick change” configuration). Further embodiments contain sealing elements that help to prevent fluid ingress into the mechanism.

According to one implementation, certain of the internal components depicted in FIGS. 5A-5C are configured to actuate rotation at the end effector 28A around axis E (as best shown in FIG. 5B), which is parallel to the longitudinal axis of the right forearm 20A. This rotation around axis E is also referred to as “tool roll.” The rotation, in one aspect, is created as follows. An actuator 180 is provided that is, in this implementation, a motor assembly 180. The motor assembly 180 is operably coupled to the motor gear 182, which is a spur gear in this embodiment. The motor gear 182 is coupled to the driven gear 184 such that rotation of the motor gear 182 causes rotation of the driven gear 184. The driven gear 184 is fixedly coupled to the roll hub 186, which is supported by a bearing 188. The roll hub 186 is fixedly coupled to the tool base interface 190, which has external threads 190A which are threadably coupled to the end effector 28A. Thus, rotation of the driven gear 184 causes rotation of the roll hub 186, which causes rotation of the tool base interface 190, which causes rotation of the end effector 28A around axis E as shown in FIG. 5B.

In one embodiment, certain of the internal components depicted in FIGS. 5A-5C are configured to actuate the end effector to open and close. This rotation of the end effector arms such that the end effector opens and closes is also called “tool drive.” The actuation, in one aspect, is created as follows.

An actuator 200 is provided that is, in this implementation, a motor assembly 200. The motor assembly 200 is operably coupled to the motor gear 202, which is a spur gear in this embodiment. The motor gear 202 is coupled to the driven gear 204 such that rotation of the motor gear 202 causes rotation of the driven gear 204. The driven gear 204 is fixedly coupled to a tool drive nut 206, which is supported by a bearing pair 208. The tool drive nut 206 has a threaded inner lumen 206A, and this threaded inner lumen 206A is threadably coupled to the lead screw 210. More specifically, the outer threads of the lead screw 210 are threadably coupled to the threads on the inner lumen 206A. The lead screw 210 is rotationally coupled to the tool base interface 190 (discussed above). More specifically, the tool base interface 190 has a square-shaped inner lumen 190A, and the distal end of the lead screw 210 has a square-shaped protrusion that fits within the inner lumen 190A, thereby coupling with the tool base interface 190. The distal end of the lead screw 210 can move translationally within the lumen 190A, but cannot rotate in relation to the tool base interface 190, so the lead screw 210 can move translationally in relation to the tool base interface 190, but cannot rotate in relation thereto.

The lead screw 210 also has an insulating sleeve 212 disposed to an external portion of the lead screw 210 and thereby plays a role in maintaining separate electrical cautery channels as will be described below. Further, the lead screw 210 has a threaded inner lumen 210A, which is threadably coupled to the tool pin 214. The tool pin 214 is operationally coupled to a known linkage mechanism within the end effector 28A such that translation of the tool pin 214 causes the grasper arms or blades to open and close. As such, actuation of gear 202 causes rotation of the driven gear 204, which rotates the tool drive nut 206. The rotation of the tool drive nut 206 causes the lead screw 210 to translate as a result of the threadable coupling of the nut 206 and the screw 210. The translation of the screw 210 causes the tool pin 214 to translate, thereby causing the end effector 28A arms or blades to open and close.

In this embodiment, these two axes of rotation are coupled. That is, if pure roll is desired, then the tool open/close drive train must match the speed of the roll train such that there is no relative angular displacement between the tool drive nut 206 and the tool base interface 190.

According to one implementation, the end effector 28A can be quickly and easily coupled to and uncoupled from the forearm 20A in the following fashion. With both the roll and drive axes fixed or held in position, the end effector 28A can be rotated, thereby coupling or uncoupling the threads 190A and 210A. That is, if the end effector 28A is rotated in one direction, the end effector 28A is coupled to the forearm 20A, and if it is rotated in the other direction, the end effector 28A is uncoupled from the forearm 20A.

In accordance with one embodiment, the forearm 20A has two independent cautery channels (referred to herein as “channel A” and “channel B”), which enables the use of either bipolar or monopolar cautery end effectors with this forearm 20A.

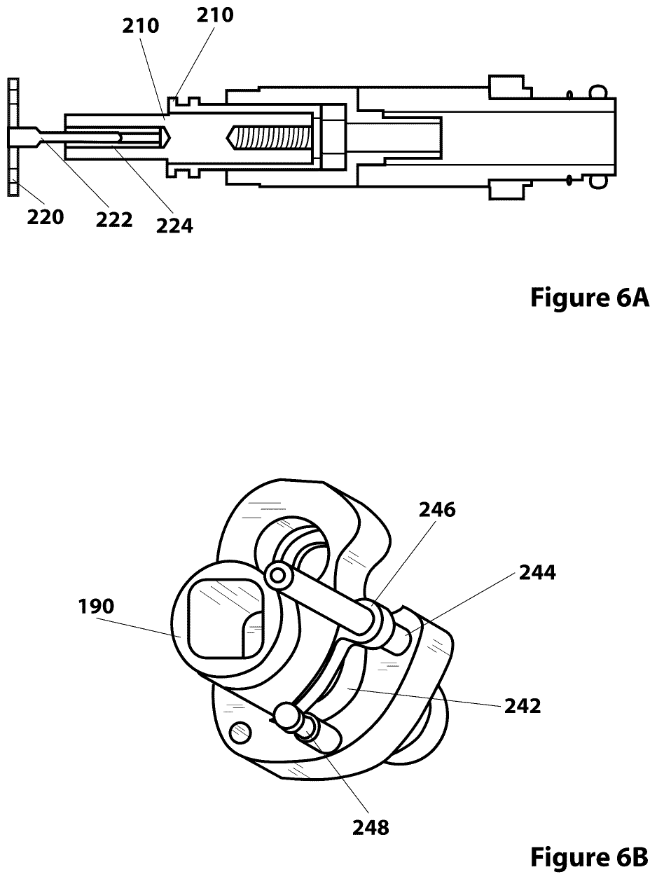

Turning to FIG. 6A, the channel A components of certain exemplary embodiments are set forth in the forearm 20A as shown. A PCB 220 is electrically coupled to lead A of a cautery power line (such as cautery line 104 discussed above) that is coupled to an external power source, such as a cautery generator. The PCB 220 is further electrically coupled to a pin 222, which is electrically coupled to socket 224 (defined in or coupled-electrically and mechanically-to a proximal end of the lead screw 210 discussed above) and is slidably positioned within the socket 224. The lead screw 210 is coupled electrically and mechanically to the end effector pin 214 as best shown in FIG. 5C. As such, energizing lead A in the cautery line 104 energizes channel A in the bipolar cautery end effector 28A. Certain embodiments of the forearm further comprise at least one insulator 225.

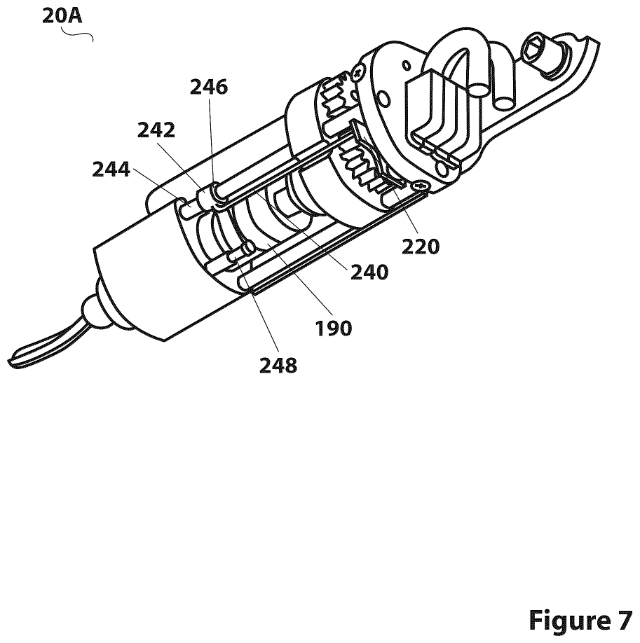

As shown in FIGS. 6B and 7, the channel B components are set forth in the forearm 20A as shown. The PCB 220 discussed above is also electrically coupled to lead B of a cautery power line (such as cautery line 104 discussed above) that is coupled to an external power source. The PCB 220 is further electrically coupled to a conducting rod 240, which is electrically coupled to a wiper 242. The wiper 242 is a tensioned component that supported on one end by a mechanical strut 244. An insulating insert 246 is positioned between the wiper 242 and the mechanical strut 244. At its free end, the wiper 242 is supported by a preloader 248. Based on this configuration, the wiper 242 is loaded or urged-like a leaf spring-against the tool base interface 190 (discussed above) and thus becomes electrically coupled to the tool base interface 190. The tool base interface 190 is mechanically coupled to the end effector 28A and electrically coupled to channel B of that end effector 28A. As such, energizing lead B in the cautery line 104 energizes channel B in the bipolar cautery end effector 28A. In exemplary embodiments, the channel A components are electrically isolated from the channel B components, and both channels are electrically isolated from the chassis to enhance patient safety.

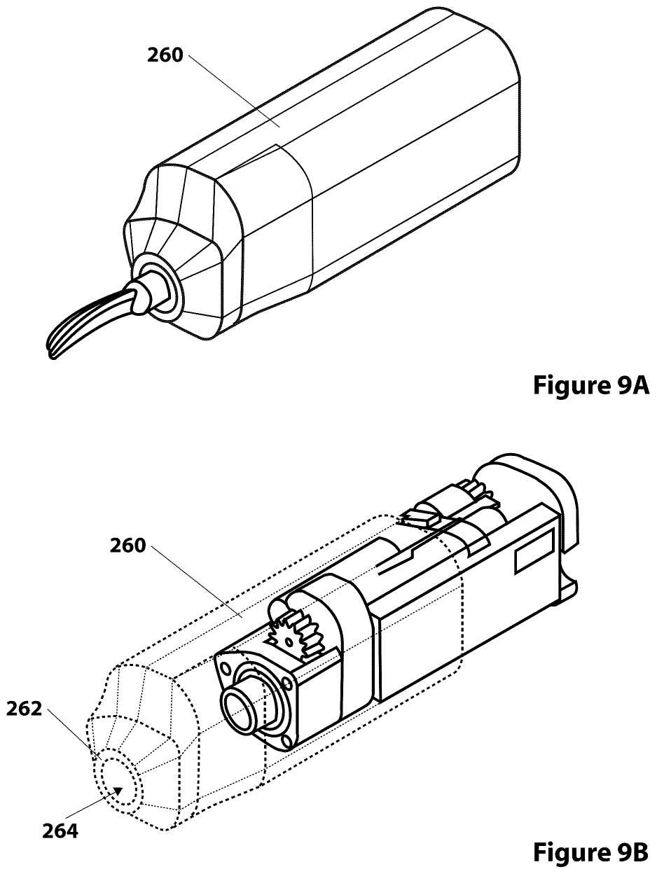

In one implementation, the forearm 20A has at least one fluidic seal interface that helps to prevent fluid ingress into the forearm 20A. One such mechanism is a monolithic single-piece housing 260 as depicted in FIGS. 9A and 9B according to one embodiment. The one-piece nature of the housing 260 greatly reduces the number of interfaces that must be sealed and thus reduces the number of interfaces where fluidic leaks are more likely to occur. The housing 260 is configured to slide over the internal components of the forearm 20A. That is, the proximal end of the housing 260 defines an opening that can be positioned over the forearm 20A (or the forearm 20A is inserted into the lumen) until the housing 260 is correctly positioned over the forearm 20A. As best shown in FIG. 9B, the housing 260 can have an O-ring 262 positioned in a groove defined in the housing 260 around the hole 264 defined in the distal end of the housing 260. The hole 264 is configured to receive the end effector 28A, which in certain embodiments is the distal end of the roll hub 186. In one embodiment, the roll hub 186 (discussed above) is positioned through the hole 264 such that the O-ring 262 is configured to be preloaded against that roll hub 186, thereby forming a fluidic seal between the housing 260 and the external surface of the hub 186, which in certain embodiments may further comprise a stainless steel ring to enhance the seal.

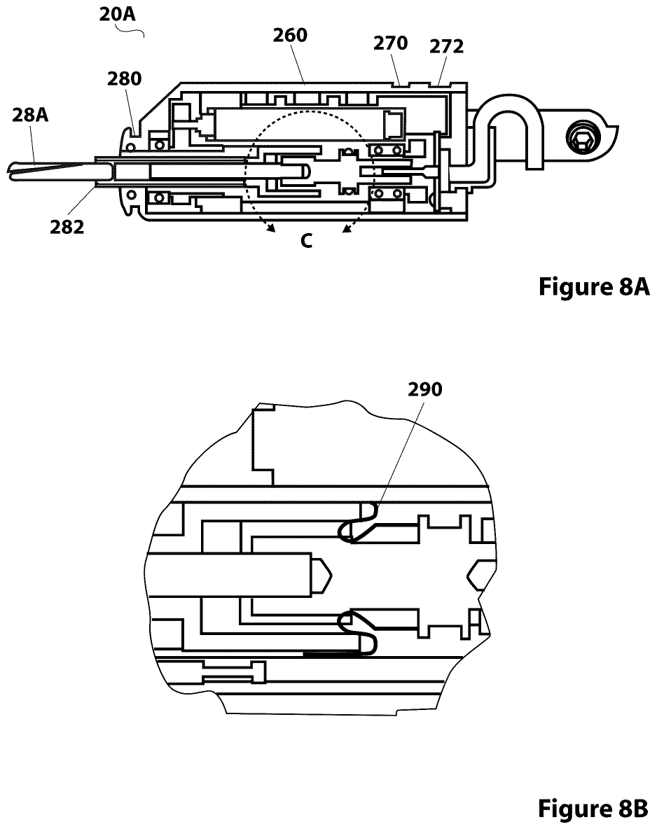

In a further embodiment as shown in FIG. 8A, the forearm 20A has two grooves 270, 272 defined in the external portion of the forearm housing 260 (as described above). The grooves 270, 272 can be configured to provide an attachment point for an outer barrier (such as the first barrier 300 described in further detail below) such that an elastic band defined in the opening of the sleeve of the inner barrier 300 can be positioned in the grooves 270, 272, thereby enhancing the coupling of the barrier 300 to the housing 260 and thus enhancing the fluidic seal. In one embodiment, the grooves 270, 272 encircle the entire forearm housing 260. Alternatively, the first barrier 300 can be bonded to the housing 260 via an adhesive or welding. In a further alternative, the housing 260 and the first barrier 300 can be fabricated as a single piece.

According to another implementation as shown in FIG. 8A, the forearm 20A housing 260 can have a groove 280 defined in the housing 260 around the hole 282 in the housing 260 through which the end effector 28A is positioned. The groove 280 can be configured to provide an attachment point for an outer barrier (such as the outer barrier 310 described in further detail below) such that an elastic band defined in the opening of the sleeve of the second barrier 310 can be positioned in the grooves 270, 272, thereby enhancing the coupling of the second barrier 310 to the housing 260 and thus enhancing the fluidic seal.

As shown in FIG. 8B, another fluidic seal can be provided according to another embodiment in the form of a flexible membrane 290 that is attached at one end to the lead screw 210 (discussed above) and at the other end to the tool base interface 190 (discussed above). More specifically, the membrane 290 is coupled to the lead screw 210 at the O-ring 292 and is coupled to the tool base interface 190 at the groove 292. In one embodiment, the membrane 290 is retained at the groove 292 with an attachment mechanism such as a cinch (not shown). This membrane 290 serves to provide a fluidic seal for the internal components of the forearm 20A against any external fluids. In one implementation, the seal is maintained whether the end effector 28A is coupled to the forearm 20A or not. Alternatively, the membrane 290 can be replaced with a metallic bellows.

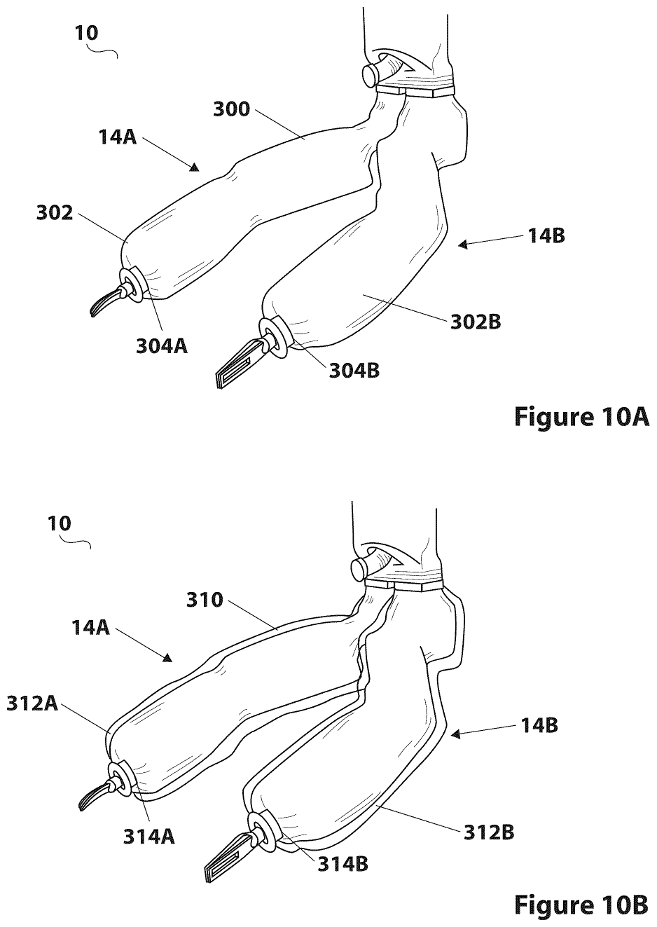

Additional fluidic seals can be provided according to certain embodiments as depicted in FIGS. 10A and 10B. As shown in FIGS. 10A and 10B, the device 10 can have two fluidically sealed barriers protecting each of the device arms 14A, 14B. The first barrier (also referred to herein as an “inner barrier”) 300 is shown in FIG. 10A, in which it is positioned around each arm and coupled at the sleeve ends 302A, 302B to the device body 12 via elastic components 304A, 304B that urge the openings in the sleeve ends 302A, 302B, thereby enhancing the fluidic seal. In the embodiment as shown, the elastic components 304A, 304B are positioned around the forearms of the arms 14A at the distal ends of the forearms. Alternatively as described in detail above with respect to FIG. 8A, the elastic components 304A, 304B can be positioned in grooves defined in the forearms (such as grooves 270, 272 described above).

In one embodiment, the inner barrier 300 is a membrane that is permanently bonded to the device 10 and is not removed for the entire operational life of the device 10. The barrier 300 is sterilized with the device 10.

The second barrier (also referred to herein as an “outer barrier”) 310 is shown in FIG. 10B, in which is positioned around each arm 14A, 14B, over the inner barrier 300 discussed above, and coupled at the sleeve ends 312A, 312B to the device body 12 via elastic components 314A, 314B that urge the openings at the sleeve ends 312A, 312B against the arms 14A, 14B, thereby enhancing the fluid seal.

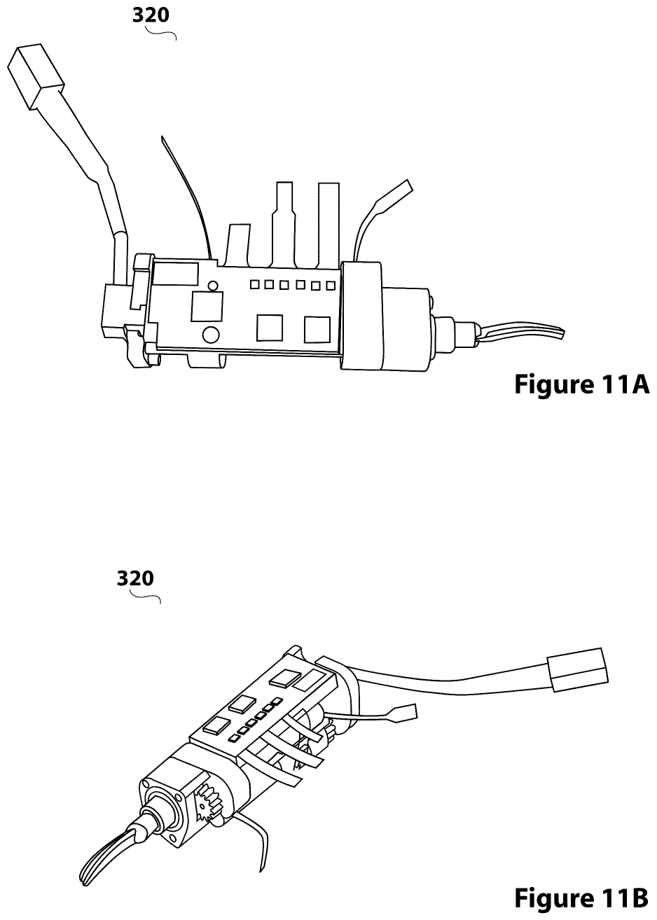

FIGS. 11A and 11B depict one embodiment of a rigid-flex PCB component 320 that can be used as the PCB component within the device embodiments described above. It is understood that the rigid-flex assembly is a known fabrication method. In one embodiment, the PCB component 320 that has been assembled using a known fabrication method, but is custom designed and fabricated.

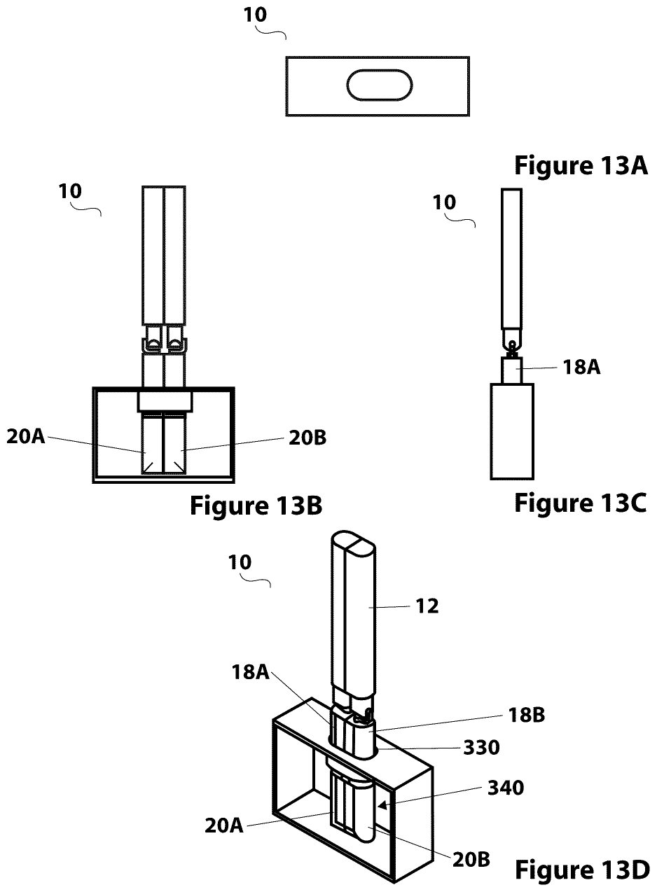

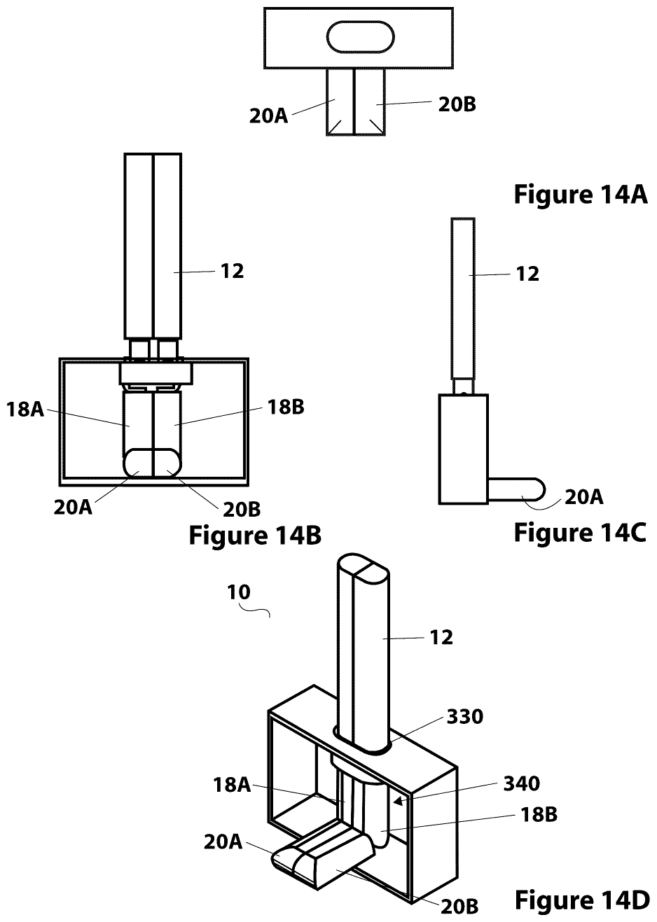

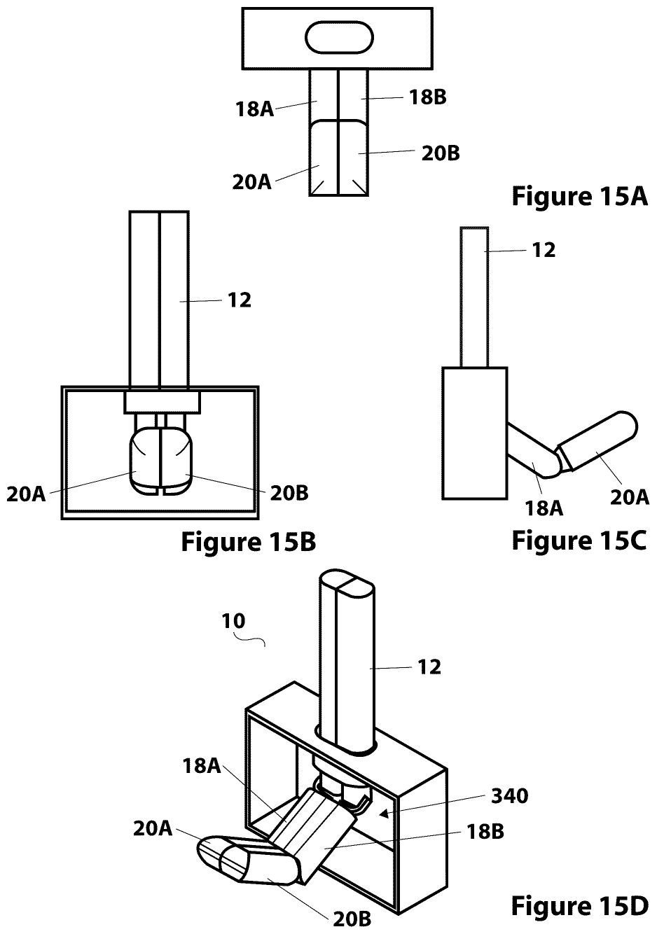

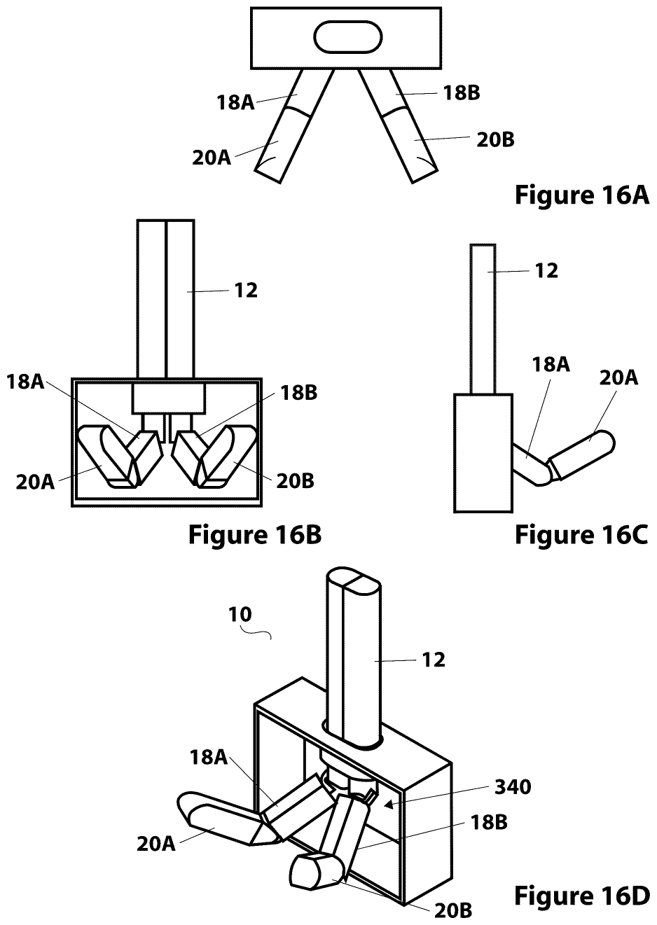

In use as shown in FIGS. 12A-17D, the device embodiments disclosed and contemplated herein are configured to have a consistent cross-section and minimal profile, thereby enhancing the ease of inserting the device through an incision and into a patient's cavity. Further, in one embodiment, the device 10 can be inserted via a specific set of steps that maintain the minimal profile and consistent cross-section in an optimal fashion. As shown in FIG. 12, the device 10 is being prepared to be inserted through the incision 330 and into the cavity 340. Note that the arms 14A, 14B of the device 10 are straight. In FIG. 13, the device 10 is inserted such that the forearms 20A, 20B are positioned in the cavity 340. As shown in FIG. 14, the forearms 20A, 20B can then be rotated as shown to maximize the amount of the device 10 that can be inserted. As the insertion continues as shown in FIG. 15, the upper arms 18A, 18B are also rotated to optimize the surgical space. At this point, the arms 14A, 14B can be moved into their operational position, first by urging them to move in opposite directions as shown in FIG. 16.

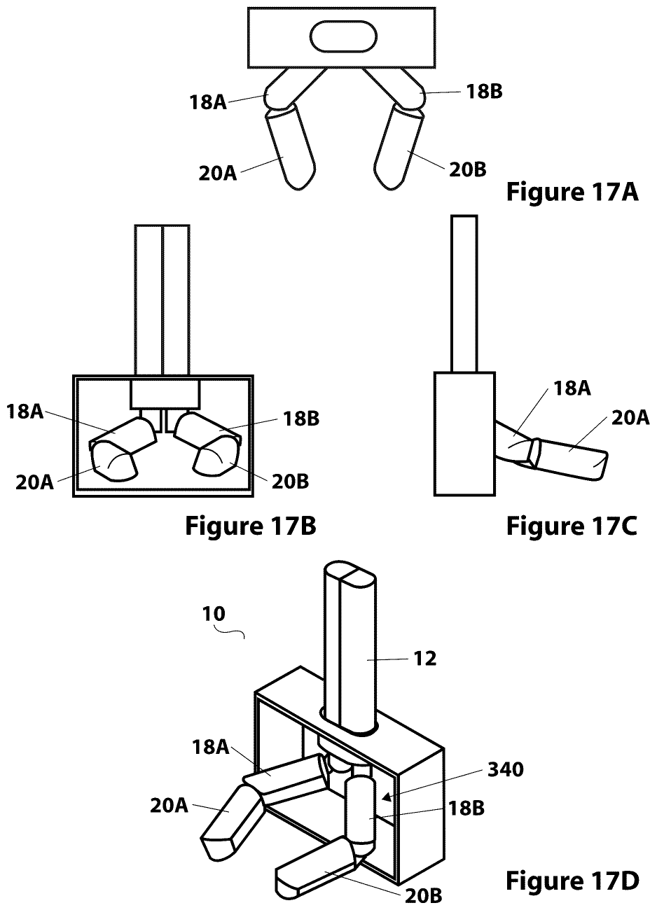

Finally, the arms 14A, 14B are rotated so that the elbows are projecting outward in FIG. 17, thereby moving the arms 14A, 14B into their preferred operational position. In exemplary embodiments, the device may be rotated and/or tilted inside the patient relative to the initial insert position, so as to provide the user with access to all four quadrants from the single insertion. Further, as is apparent from the insertion of the device depicted in FIGS. 12A-17D, the arms of the device are inserted in parallel, rather than sequentially, as had been the case in prior surgical robotic devices.

In one implementation, the device 10 has at least one camera that is used in conjunction with the device 10. For example, a camera (not shown) such as a camera having two degrees of freedom (a pan-and-tilt camera) having digital zoom could be used. In one embodiment, it is inserted through the camera lumen 32 defined in the proximal end of the device body 12 as best shown in FIG. 1C. According to one implementation, the camera can be controlled by the user or surgeon using a foot controller and would be easy to remove, clean, and re-insert during a procedure. In another embodiment, the camera can be a standard laparoscope inserted through the same incision, through the lumen 32, or through a different incision.

While multiple embodiments are disclosed, still other embodiments of the present invention will become apparent to those skilled in the art from the following detailed description, which shows and describes illustrative embodiments of the invention. As will be realized, the invention is capable of modifications in various obvious aspects, all without departing from the spirit and scope of the present invention. Accordingly, the drawings and detailed description are to be regarded as illustrative in nature and not restrictive.

Although the present invention has been described with reference to preferred embodiments, persons skilled in the art will recognize that changes may be made in form and detail without departing from the spirit and scope of the invention.

1. A surgical robotic device for performing surgery on a patient, the device comprising:
a) an elongate device body sized to be positioned through an incision of a patient;
b) a first robotic arm rotatably coupled to a distal end of the elongate device body and comprising a first upper arm segment, a first forearm housing, and a first operational component supported by the first forearm housing;
c) a second robotic arm rotatably coupled to the distal end of the elongate device body and comprising a second upper arm segment, a second forearm housing, and a second operational component supported by the second forearm housing;
d) at least one first actuator disposed within the first forearm housing and configured to actuate the first operational component;
e) at least one second actuator disposed within the second forearm housing and configured to actuate the second operational component; and
f) at least one fluidic seal interface associated with each of the first forearm housing and the second forearm housing, the at least one fluidic seal interface being configured to inhibit fluid ingress into a corresponding one of the first forearm housing or the second forearm housing.

2. The surgical robotic device of claim 1, wherein each of the first forearm housing and the second forearm housing comprises a monolithic single-piece housing configured to enclose internal components of the corresponding first robotic arm or second robotic arm.

3. The surgical robotic device of claim 1, wherein each of the first forearm housing and the second forearm housing comprises a distal opening configured to receive at least a portion of a roll hub coupled to the corresponding first operational component or second operational component.

4. The surgical robotic device of claim 3, wherein the at least one fluidic seal interface associated with each of the first forearm housing and the second forearm housing comprises an O-ring positioned in a groove defined in the corresponding first forearm housing or second forearm housing around the distal opening.

5. The surgical robotic device of claim 4, wherein each O-ring is configured to be preloaded against the corresponding roll hub to form a fluidic seal between the corresponding first forearm housing or second forearm housing and an external surface of the corresponding roll hub.

6. The surgical robotic device of claim 1, wherein each of the first forearm housing and the second forearm housing comprises one or more grooves defined in an external portion of the corresponding first forearm housing or second forearm housing, the one or more grooves being configured to receive an elastic component of a barrier sleeve to enhance a fluidic seal between the barrier sleeve and the corresponding first forearm housing or second forearm housing.

7. The surgical robotic device of claim 1, wherein the at least one fluidic seal interface associated with each of the first forearm housing and the second forearm housing comprises a flexible membrane coupled between a lead screw and a tool base interface of the corresponding first robotic arm or second robotic arm.

8. The surgical robotic device of claim 7, wherein the flexible membrane comprises a metallic bellows.

9. The surgical robotic device of claim 1, further comprising an inner barrier positioned around each of the first robotic arm and the second robotic arm and coupled to the elongate device body by elastic components configured to enhance a fluidic seal.

10. The surgical robotic device of claim 9, further comprising an outer barrier positioned around each of the first robotic arm and the second robotic arm and over the inner barrier, the outer barrier being coupled to the elongate device body by elastic components configured to urge openings of the outer barrier against the first robotic arm and the second robotic arm.

11. The surgical robotic device of claim 1, wherein the first operational component comprises a first removable end effector coupled to the first forearm housing and the second operational component comprises a second removable end effector coupled to the second forearm housing, and wherein at least one of the first removable end effector and the second removable end effector is threadably coupled to a tool base interface and a tool pin of the corresponding first robotic arm or second robotic arm.

12. The surgical robotic device of claim 1, wherein at least one of the first forearm housing and the second forearm housing contains a first cautery channel and a second cautery channel independent from the first cautery channel, and wherein the first cautery channel and the second cautery channel are electrically isolated from each other and from a chassis of the surgical robotic device.

13. A surgical robotic device for performing surgery on a patient, the device comprising:
a) an elongate device body sized to be positioned through an incision of a patient, the elongate device body comprising a proximal end, a distal end, and a camera lumen defined in the proximal end and extending at least partially between the proximal end and the distal end;
b) a first robotic arm rotatably coupled to the distal end of the elongate device body and comprising a first upper arm segment, a first forearm housing, and a first operational component supported by the first forearm housing;
c) a second robotic arm rotatably coupled to the distal end of the elongate device body and comprising a second upper arm segment, a second forearm housing, and a second operational component supported by the second forearm housing; and
d) a camera removably positionable through the camera lumen and configured to provide visualization of a surgical site during use of the first robotic arm and the second robotic arm.

14. The surgical robotic device of claim 13, wherein the camera comprises a pan-and-tilt camera having two degrees of freedom.

15. The surgical robotic device of claim 13, wherein the camera is configured to be removable from the camera lumen, cleanable, and reinsertable through the camera lumen during a surgical procedure.

16. The surgical robotic device of claim 13, wherein the camera is configured to provide digital zoom.

17. A surgical robotic device for performing surgery on a patient, the device comprising:
a) an elongate device body sized to be positioned through an incision of a patient;
b) a first robotic arm rotatably coupled to a distal end of the elongate device body and comprising a first upper arm segment, a first forearm housing, and a first operational component supported by the first forearm housing;
c) a second robotic arm rotatably coupled to the distal end of the elongate device body and comprising a second upper arm segment, a second forearm housing, and a second operational component supported by the second forearm housing; and
d) first and second cautery channels disposed at least partially within at least one of the first forearm housing and the second forearm housing and electrically coupled to a corresponding one of the first operational component and the second operational component,
wherein the first and second cautery channels are electrically isolated from each other and are configured to enable use of either a bipolar cautery operational component or a monopolar cautery operational component with a corresponding one of the first forearm housing and the second forearm housing.

18. The surgical robotic device of claim 17, wherein the first cautery channel comprises a first electrical path extending through a printed circuit board, a pin, a socket, a lead screw, and an operational component pin disposed within the at least one of the first forearm housing and the second forearm housing.

19. The surgical robotic device of claim 17, wherein the second cautery channel comprises a second electrical path extending through a printed circuit board, a conducting rod, a wiper, and an operational component interface disposed within the at least one of the first forearm housing and the second forearm housing.

20. The surgical robotic device of claim 17, wherein the first cautery channel and the second cautery channel are electrically isolated from a chassis of the surgical robotic device.
